(19) United States
(12) Patent Application Publication
(10) Pub. No.: US 2023/0410250 A1
(43) Pub. Date: Dec. 21, 2023

(54) IMAGING METHODS USING RADIATION DETECTORS

(71) Applicant: SHENZHEN XPECTVISION TECHNOLOGY CO., LTD., Shenzhen (CN)

(72) Inventors: Peiyan CAO, Shenzhen (CN); Yurun LIU, Shenzhen (CN)

(21) Appl. No.: 18/238,095

(22) Filed: Aug. 25, 2023

Related U.S. Application Data
(63) Continuation of application No. PCT/CN2021/079217, filed on Mar. 5, 2021.

(51) Int. Cl. G06T 3/40; G06T 7/13

(52) U.S. Cl. CPC G06T 3/4038; G06T 2200/32; G06T 2207/10072; G06T 7/13

(57) ABSTRACT

Disclosed herein is a method, comprising: for i=1, . . . , N, one by one, exposing a radiation detector to a radiation beam (i) thereby causing the radiation detector to capture a partial image (i) of the radiation beam (i), wherein N is an integer greater than 1; for i=1, . . . , N, determining, in the partial image (i), Mi pinpointing picture elements of a boundary image (i) of a boundary (i) of the radiation beam (i), wherein Mi is a positive integer; and stitching the partial images (i), i=1, . . . , N resulting in a combined image based on the Mi (i=1, . . . , N) pinpointing picture elements.

### TECHNICAL FIELD

The disclosure herein relates to imaging methods using radiation detectors.

### BACKGROUND

A radiation detector is a device that measures a property of a radiation. Examples of the property may include a spatial distribution of the intensity, phase, and polarization of the radiation. The radiation may be one that has interacted with an object. For example, the radiation measured by the radiation detector may be a radiation that has penetrated the object. The radiation may be an electromagnetic radiation such as infrared light, visible light, ultraviolet light, X-ray or γ-ray. The radiation may be of other types such as α-rays and β-rays. An image sensor of an imaging system may include multiple radiation detectors.

### SUMMARY

Disclosed herein is a method, comprising: for i=1, . . . , N, one by one, exposing a radiation detector to a radiation beam (i) thereby causing the radiation detector to capture a partial image (i) of the radiation beam (i), wherein N is an integer greater than 1; for i=1, . . . , N, determining, in the partial image (i), Mi pinpointing picture elements of a boundary image (i) of a boundary (i) of the radiation beam (i), wherein Mi is a positive integer; and stitching the partial images (i), i=1, . . . , N resulting in a combined image based on the Mi (i=1, . . . , N) pinpointing picture elements.

In an aspect, for i=1, . . . , N, the boundary image (i) is a closed line.

In an aspect, for i=1, . . . , N, the boundary image (i) is a rectangle.

In an aspect, for i=1, . . . , N, the Mi pinpointing picture elements comprise a pinpointing picture element (i, 1), a pinpointing picture element (i, 2), a pinpointing picture element (i, 3), a pinpointing picture element (i, 4), and a pinpointing corner picture element (i), and wherein for i=1, . . . , N, the pinpointing corner picture element (i) is on both (A) a straight line going through the pinpointing picture element (i, 1) and the pinpointing picture element (i, 2), and (B) a straight line going through the pinpointing picture element (i, 3) and the pinpointing picture element (i, 4).

In an aspect, for i=1, . . . , N, the boundary image (i) is not a closed line.

In an aspect, for i=1, . . . , N, intensity of radiation gradually falls when moving from inside the radiation beam (i) to outside the radiation beam (i) across the boundary (i) of the radiation beam (i).

In an aspect, for i=1, . . . , N−1, a region (i) of the partial image (i) bounded by the boundary image (i) overlaps a region (i+1) of the partial image (i+1) bounded by the boundary image (i+1).

In an aspect, for i=1, . . . , N, values of picture elements of the partial image (i) outside the boundary image (i) as pinpointed by the Mi pinpointing picture elements are not used in determining values of picture elements of the combined image.

In an aspect, for i=1, . . . , N, values of some picture elements of the partial image (i) outside the boundary image (i) as pinpointed by the Mi pinpointing picture elements are used in determining values of picture elements of the combined image.

Disclosed herein is a method, comprising: exposing a first radiation detector to a radiation beam thereby causing the first radiation detector to capture a first beam image of the radiation beam; and determining, in the first beam image, M1 pinpointing picture elements of a first boundary image of a boundary of the radiation beam, wherein M1 is a positive integer.

In an aspect, the first boundary image is a closed line.

In an aspect, the first boundary image is a rectangle.

In an aspect, the M1 pinpointing picture elements comprise a first pinpointing picture element, a second pinpointing picture element, a third pinpointing picture element, a fourth pinpointing picture element, and a pinpointing corner picture element, and wherein the pinpointing corner picture element is on both (A) a first straight line going through the first and second pinpointing picture elements, and (B) a second straight line going through the third and fourth pinpointing picture elements.

In an aspect, the first boundary image is not a closed line.

In an aspect, intensity of radiation gradually falls when moving from inside the radiation beam to outside the radiation beam across the boundary of the radiation beam.

In an aspect, the method further comprises: exposing a second radiation detector to the radiation beam thereby causing the second radiation detector to capture a second beam image of the radiation beam; and determining, in the second beam image, M2 pinpointing picture elements of a second boundary image of the boundary of the radiation beam, wherein M2 is a positive integer.

Disclose herein is an apparatus, comprising a first radiation detector configured to (A) capture a first beam image of a radiation beam in response to the first radiation detector being exposed to the radiation beam and (B) determine, in the first beam image, M1 pinpointing picture elements of a first boundary image of a boundary of the radiation beam, wherein M1 is a positive integer.

In an aspect, the first boundary image is a closed line.

In an aspect, intensity of radiation gradually falls when moving from inside the radiation beam to outside the radiation beam across the boundary of the radiation beam.

In an aspect, the apparatus further comprises a second radiation detector configured to (A) capture a second image of the radiation beam in response to the second radiation detector being exposed to the radiation beam and (B) determine, in the second beam image, M2 pinpointing picture elements of a second boundary image of the boundary of the radiation beam, wherein M2 is a positive integer.

### BRIEF DESCRIPTION OF FIGURES

FIG. 1 schematically shows a radiation detector, according to an embodiment.

FIG. 2A schematically shows a simplified cross-sectional view of the radiation detector, according to an embodiment.

FIG. 2B schematically shows a detailed cross-sectional view of the radiation detector, according to an embodiment.

FIG. 2C schematically shows a detailed cross-sectional view of the radiation detector, according to an alternative embodiment.

FIG. 3A schematically shows an imaging system, according to an embodiment.

FIG. 3B-FIG. 3C show an image captured by the imaging system, according to an embodiment.

FIG. 3D shows a flowchart summarizing and generalizing an operation of the imaging system, according to an embodiment.

FIG. 3E-FIG. 3F show the imaging system, according to an alternative embodiment.

FIG. 3G shows the imaging system, according to yet another alternative embodiment.

FIG. 4A-FIG. 4G show an operation of the imaging system using multiple exposures, according to an embodiment.

FIG. 5 shows a flowchart summarizing and generalizing an operation of the imaging system of FIG. 4A-FIG. 4G, according to an embodiment.

### DETAILED DESCRIPTION

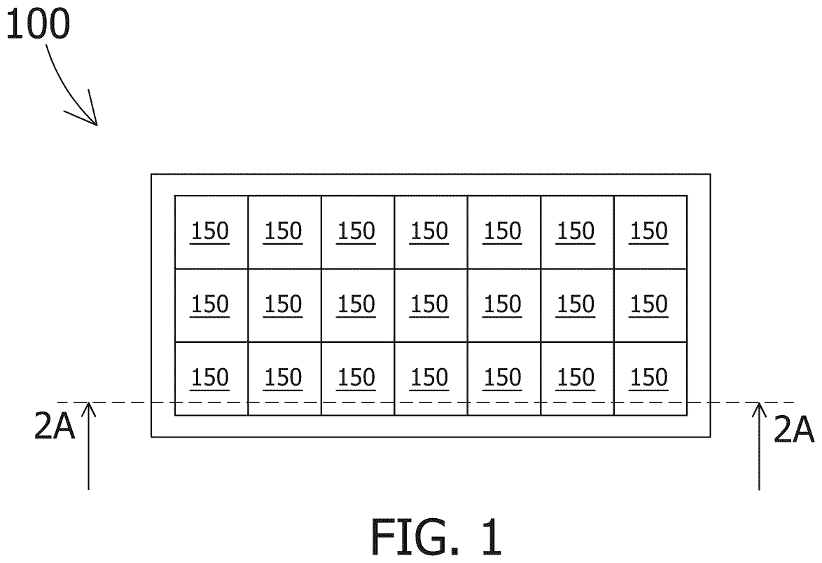

FIG. 1 schematically shows a radiation detector 100, as an example. The radiation detector 100 may include an array of pixels 150 (also referred to as sensing elements 150). The array may be a rectangular array (as shown in FIG. 1), a honeycomb array, a hexagonal array, or any other suitable array. The array of pixels 150 in the example of FIG. 1 has 21 pixels 150 arranged in 3 rows and 7 columns. In general, the array of pixels 150 may have any number of pixels 150 arranged in any way.

A radiation may include particles such as photons (electromagnetic waves) and subatomic particles (e.g., neutrons, protons, electrons, alpha particles, etc.) Each pixel 150 may be configured to detect radiation incident thereon and may be configured to measure a characteristic (e.g., the energy of the particles, the wavelength, and the frequency) of the incident radiation. The measurement results for the pixels 150 of the radiation detector 100 constitute an image of the radiation incident on the pixels. It may be said that the image is of an object or a scene which the incident radiation come from.

Each pixel 150 may be configured to count numbers of particles of radiation incident thereon whose energy falls in a plurality of bins of energy, within a period of time. All the pixels 150 may be configured to count the numbers of particles of radiation incident thereon within a plurality of bins of energy within the same period of time. When the incident particles of radiation have similar energy, the pixels 150 may be simply configured to count numbers of particles of radiation incident thereon within a period of time, without measuring the energy of the individual particles of radiation.

Each pixel 150 may have its own analog-to-digital converter (ADC) configured to digitize an analog signal representing the energy of an incident particle of radiation into a digital signal, or to digitize an analog signal representing the total energy of a plurality of incident particles of radiation into a digital signal. The pixels 150 may be configured to operate in parallel. For example, when one pixel 150 measures an incident particle of radiation, another pixel 150 may be waiting for a particle of radiation to arrive. The pixels 150 may not have to be individually addressable.

The radiation detector 100 described here may have applications such as in an X-ray telescope, X-ray mammography, industrial X-ray defect detection, X-ray microscopy or microradiography, X-ray casting inspection, X-ray non-destructive testing, X-ray weld inspection, X-ray digital subtraction angiography, etc. It may be suitable to use this radiation detector 100 in place of a photographic plate, a photographic film, a PSP plate, an X-ray image intensifier, a scintillator, or another semiconductor X-ray detector.

An image sensor of an imaging system (not shown) may include multiple radiation detectors 100. In an embodiment, all the pixels 150 of the radiation detectors 100 of the image sensor may be coplanar (i.e., a plane intersects all the pixels 150 of all the radiation detectors 100. In an alternative embodiment, for each radiation detector 100 of the image sensor, the pixels 150 of the radiation detector 100 may be coplanar, but all the pixels 150 of all the radiation detectors 100 of the image sensor may be not coplanar. For example, the pixels 150 of a first radiation detector 100 of the image sensor may be on a first plane, but the pixels 150 of a second radiation detector 100 of the image sensor may be on a second plane different from the first plane. The first plane and the second plane may be parallel to each other, or may be not parallel to each other. For example, the radiation detectors 100 of the image sensor may be arranged on an inner surface (i.e., concave surface) of a parabola.

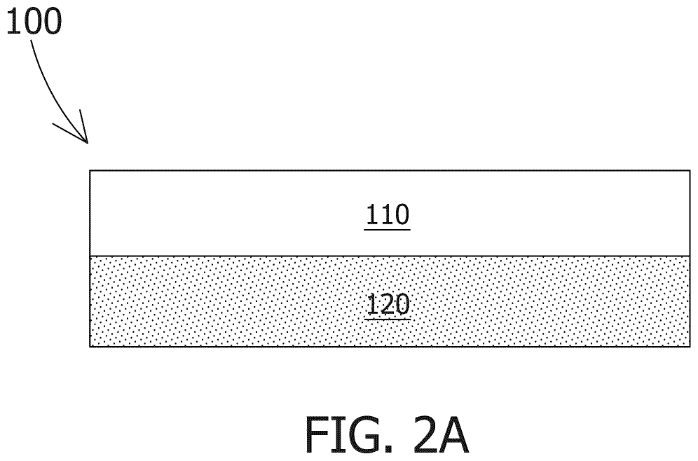

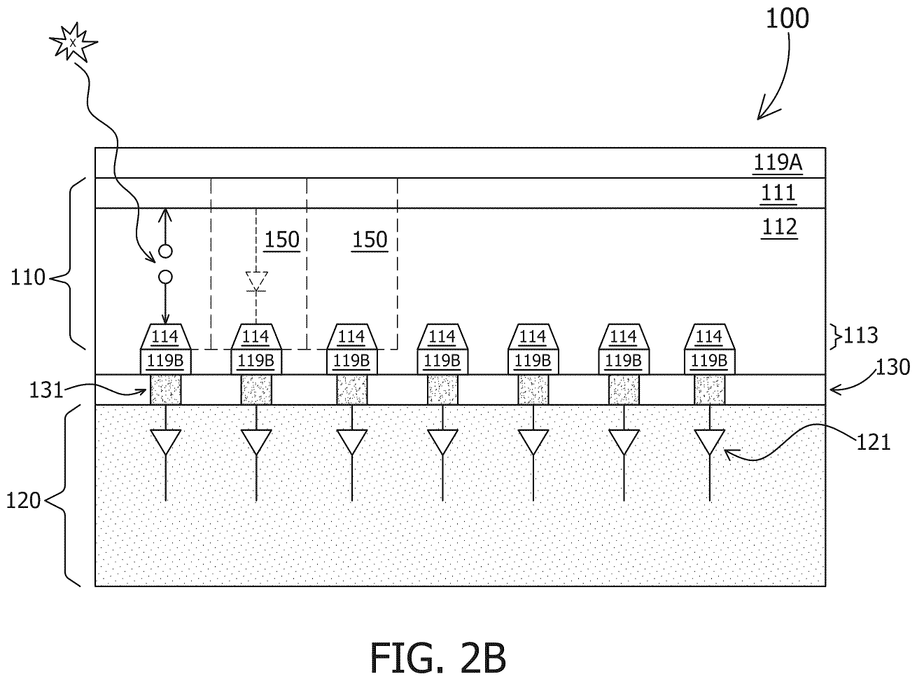

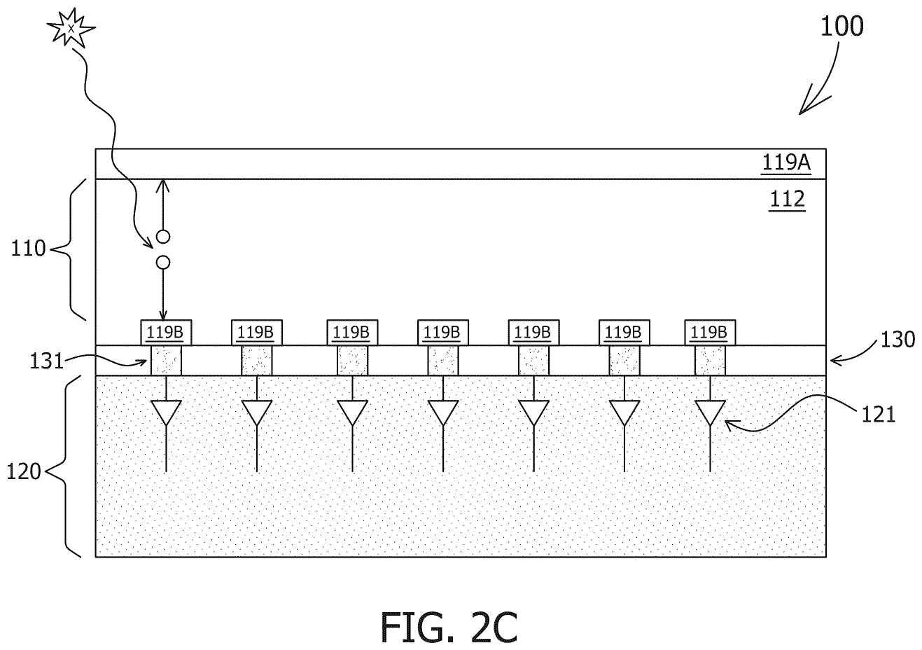

FIG. 2A schematically shows a simplified cross-sectional view of the radiation detector 100 of FIG. 1 along a line 2A-2A, according to an embodiment. More specifically, the radiation detector 100 may include a radiation absorption layer 110 and an electronics layer 120. The electronics layer 120 may include one or more application-specific integrated circuit (ASIC) chips for processing or analyzing electrical signals which incident radiation generates in the radiation absorption layer 110. The radiation detector 100 may or may not include a scintillator (not shown). The radiation absorption layer 110 may comprise a semiconductor material such as silicon, germanium, GaAs, CdTe, CdZnTe, or a combination thereof. The semiconductor material may have a high mass attenuation coefficient for the radiation of interest.

FIG. 2B schematically shows a detailed cross-sectional view of the radiation detector 100 of FIG. 1 along the line 2A-2A, as an example. More specifically, the radiation absorption layer 110 may include one or more diodes (e.g., p-i-n or p-n) formed by a first doped region 111 and one or more discrete regions 114 of a second doped region 113. The second doped region 113 may be separated from the first doped region 111 by an optional intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example of FIG. 2B, each of the discrete regions 114 of the second doped region 113 forms a diode with the first doped region 111 and the optional intrinsic region 112. Namely, in the example in FIG. 2B, the radiation absorption layer 110 has a plurality of diodes (more specifically, FIG. 2B shows 7 diodes corresponding to 7 pixels 150 of one row in the array of FIG. 1, of which only 2 pixels 150 are labeled in FIG. 2B for simplicity). The plurality of diodes have an electrode 119A as a shared (common) electrode. The first doped region 111 may also have discrete portions.

The electronics layer 120 may include an electronic system 121 suitable for processing or interpreting signals generated by the radiation incident on the radiation absorption layer 110. The electronic system 121 may include an analog circuitry such as a filter network, amplifiers, integrators, and comparators, or a digital circuitry such as a microprocessor, and memory. The electronic system 121 may include one or more ADCs. The electronic system 121 may include components shared by the pixels 150 or components dedicated to a single pixel 150. For example, the electronic system 121 may include an amplifier dedicated to each pixel 150 and a microprocessor shared among all the pixels 150. The electronic system 121 may be electrically connected to the pixels 150 by vias 131. Space among the vias may be filled with a filler material 130, which may increase the mechanical stability of the connection of the electronics layer 120 to the radiation absorption layer 110. Other bonding techniques are possible to connect the electronic system 121 to the pixels 150 without using the vias 131.

When radiation from the radiation source (not shown) hits the radiation absorption layer 110 including diodes, particles of the radiation may be absorbed and generate one or more charge carriers (e.g., electrons, holes) by a number of mechanisms. The charge carriers may drift to the electrodes of one of the diodes under an electric field. The field may be an external electric field. The electrical contact 119B may include discrete portions each of which is in electrical contact with the discrete regions 114. The term “electrical contact” may be used interchangeably with the word “electrode.” In an embodiment, the charge carriers may drift in directions such that the charge carriers generated by a single particle of the radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of the radiation incident around the footprint of one of these discrete regions 114 are not substantially shared with another of these discrete regions 114. A pixel 150 associated with a discrete region 114 may be a space around the discrete region 114 in which substantially all (more than 98%, more than 99.5%, more than 99.9%, or more than 99.99% of) charge carriers generated by a particle of the radiation incident therein flow to the discrete region 114. Namely, less than 2%, less than 1%, less than 0.1%, or less than 0.01% of these charge carriers flow beyond the pixel 150.

FIG. 2C schematically shows a detailed cross-sectional view of the radiation detector 100 of FIG. 1 along the line 2A-2A, as another example. More specifically, the radiation absorption layer 110 may include a resistor of a semiconductor material such as silicon, germanium, GaAs, CdTe, CdZnTe, or a combination thereof, but does not include a diode. The semiconductor material may have a high mass attenuation coefficient for the radiation of interest. In an embodiment, the electronics layer 120 of FIG. 2C may be similar to the electronics layer 120 of FIG. 2B in terms of structure and function.

When the radiation hits the radiation absorption layer 110 including the resistor but not diodes, it may be absorbed and generate one or more charge carriers by a number of mechanisms. A particle of the radiation may generate 10 to 100,000 charge carriers. The charge carriers may drift to the electrical contacts 119A and 119B under an electric field. The electric field may be an external electric field. The electrical contact 119B includes discrete portions. In an embodiment, the charge carriers may drift in directions such that the charge carriers generated by a single particle of the radiation are not substantially shared by two different discrete portions of the electrical contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of the radiation incident around the footprint of one of these discrete portions of the electrical contact 119B are not substantially shared with another of these discrete portions of the electrical contact 119B. A pixel 150 associated with a discrete portion of the electrical contact 119B may be a space around the discrete portion in which substantially all (more than 98%, more than 99.5%, more than 99.9% or more than 99.99% of) charge carriers generated by a particle of the radiation incident therein flow to the discrete portion of the electrical contact 119B. Namely, less than 2%, less than 0.5%, less than 0.1%, or less than 0.01% of these charge carriers flow beyond the pixel associated with the one discrete portion of the electrical contact 119B.

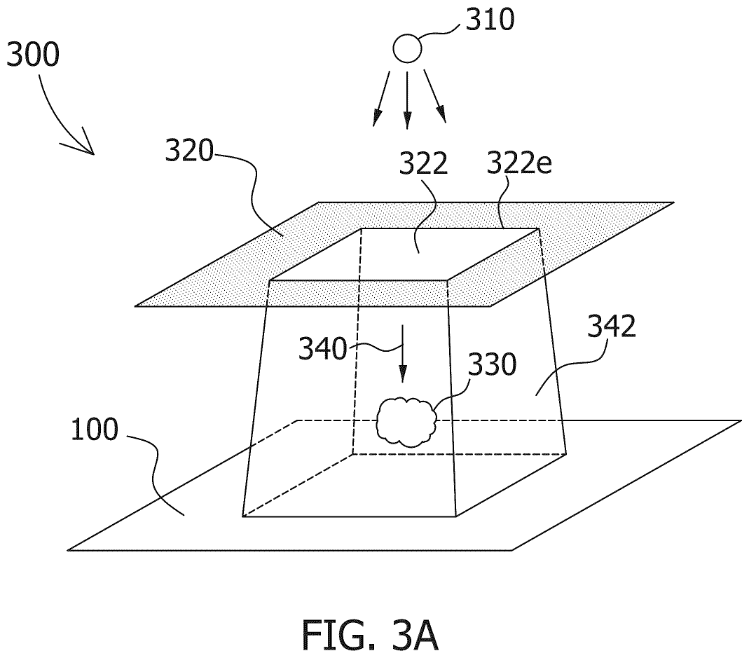

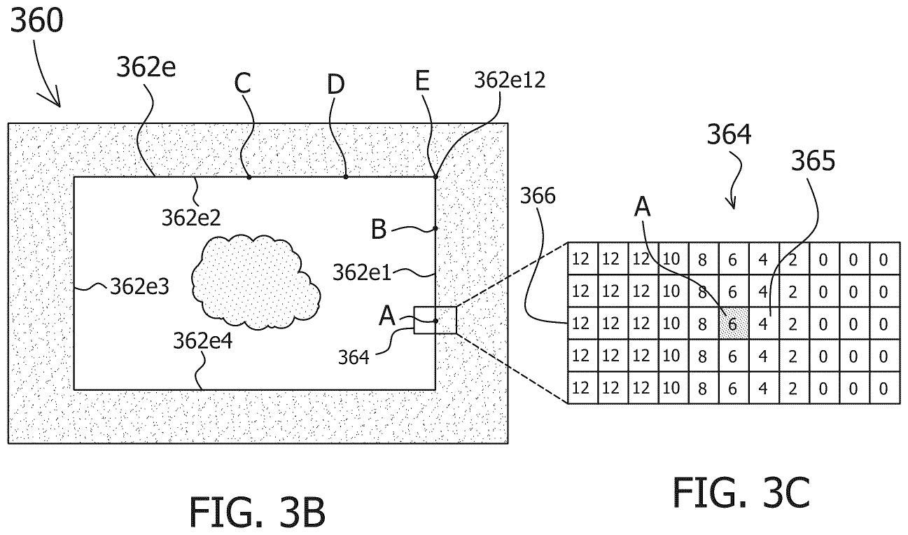

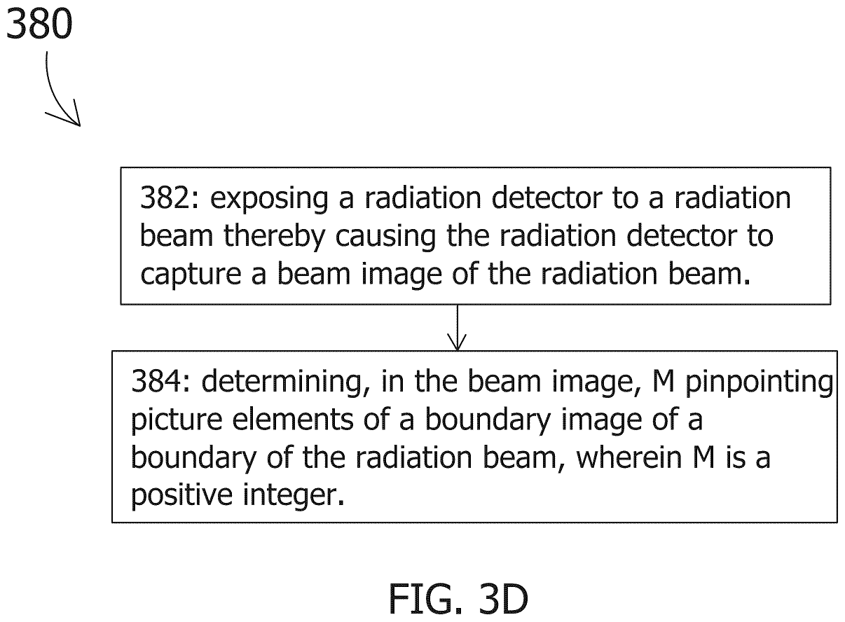

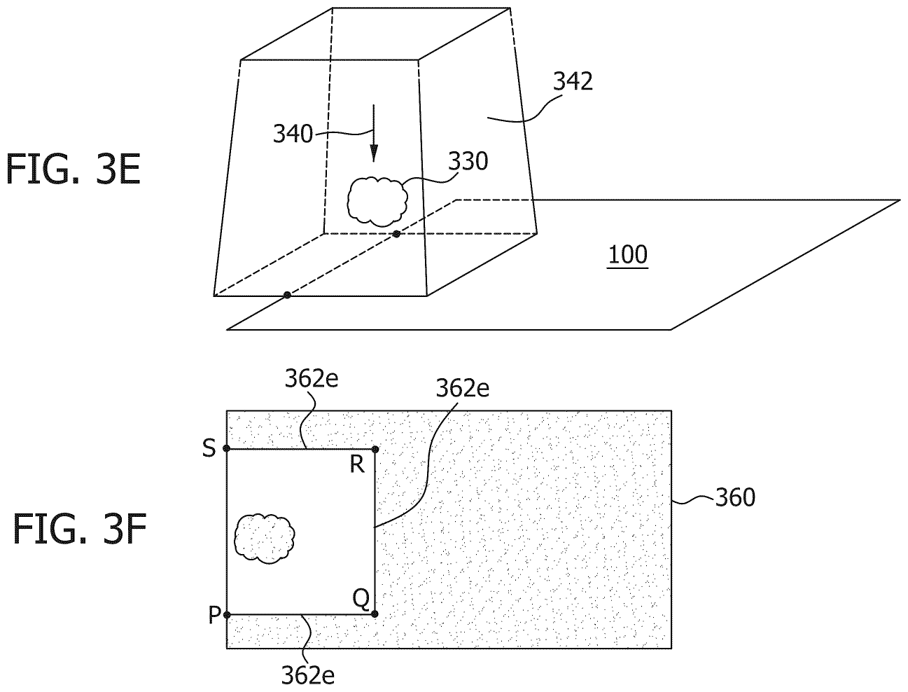

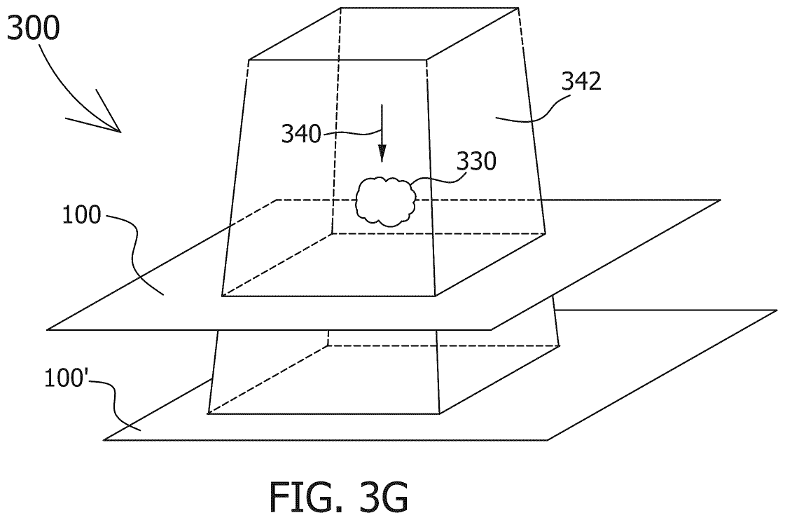

FIG. 3A schematically shows an imaging system 300, according to an embodiment. In an embodiment, the imaging system 300 may include the radiation detector 100, a radiation source 310, and a mask 320. In an embodiment, the absorption layer 110 (FIG. 2A) of the radiation detector 100 may face the radiation source 310 and the mask 320 (i.e., the absorption layer 110 is between the mask 320 and the electronics layer 120 of the radiation detector 100).

In an embodiment, the operation of the imaging system 300 may be as follows. An object 330 may be positioned between the mask 320 and the radiation detector 100. The radiation source 310 may generate radiation toward the mask 320. In an embodiment, the portion of the radiation from the radiation source 310 incident on a mask window 322 of the mask 320 may be allowed to pass through the mask 320 (for example, the mask window 322 may be not opaque to the radiation), while the portion of the radiation from the radiation source 310 incident on other parts of the mask 320 may be blocked. As a result, after passing through the mask window 322 of the mask 320, the radiation from the radiation source 310 becomes a radiation beam represented by an arrow 340 (hence thereafter the radiation beam may be referred to as the radiation beam 340).

In an embodiment, radiation particles of the radiation beam 340 some of which have penetrated the object 330 may hit the absorption layer 110 (FIG. 2A) of the radiation detector 100 causing the radiation detector 100 to capture a beam image 360 (FIG. 3B) of the radiation beam 340. In an embodiment, the mask window 322 of the mask 320 may have a rectangular shape. As a result, the radiation beam 340 may have the shape of a truncated pyramid having 4 sides which form a boundary 342 of the radiation beam 340.

In an embodiment, with reference to FIG. 3A-FIG. 3B, an image 362e in the beam image 360 of the edge (perimeter) 322e of the mask window 322 may be a rectangle having four sides 362e1, 362e2, 362e3, and 362e4. The image 362e may be considered the image of the boundary 342 of the radiation beam 340. As a result, the image 362e may also be called the boundary image 362e.

FIG. 3C shows contents of a portion 364 of the beam image 360 in terms of picture elements and their values as an example. Each picture element of the beam image 360 corresponds to a pixel 150 (FIG. 1) and may be represented by a rectangular box. The value in a box indicates the intensity of radiation of the radiation beam 340 incident on the corresponding pixel 150. For example, a value of zero in a box of FIG. 3C indicates that the pixel 150 corresponding to the picture element represented by the box receives no incident radiation particles from the radiation beam 340.

In an embodiment, with reference to FIG. 3A-FIG. 3C, the determination of a pinpointing corner picture element E in the beam image 360 where the north east corner 362e12 of the boundary image 362e is supposed to be may start with determining in the beam image 360 a pinpointing picture element A through which the side 362e1 of the boundary image 362e is supposed to pass. In an embodiment, the determination of the pinpointing picture element A may be as follows. Firstly, a row 366 of picture elements in the beam image 360 intersecting the side 362e1 of the boundary image 362e may be chosen.

In an embodiment, the radiation source 310 and the edge 322e of the mask window 322 (FIG. 3A) may be such that intensity of radiation gradually falls when moving from inside the radiation beam 340 to outside the radiation beam 340 across the boundary 342 of the radiation beam 340. As a result, when moving from left to right in the row 366 across the side 362e1 of the boundary image 362e (FIG. 3C), the values of picture elements gradually fall from 12 to 0. The specific picture element values of 0, 2, . . . , and 12 are chosen for illustration only.

In an embodiment, the pinpointing picture element A may be determined to be a picture element of the row 366 having a value which is the average value of (A) the maximum picture element value before the picture element value drop (i.e., 12) and (B) the minimum picture element value after the picture element value drop (i.e., 0). So, the average value is (12+0)/2=6. As a result, the pinpointing picture element A of the boundary image 362e may be determined to be the picture element represented by the grayed-out box as shown in FIG. 3C.

In an embodiment, the determination of the pinpointing corner picture element E may further include determining in the beam image 360 (1) a pinpointing picture element B through which the side 362e1 of the boundary image 362e is supposed to pass, and (2) picture elements C and D through both of which the side 362e2 of the boundary image 362e is supposed to pass. In an embodiment, the determinations of the pinpointing picture elements B, C, and D may be similar to the determination of the pinpointing picture element A described above. Next, in an embodiment, the pinpointing corner picture element E may be determined to be a picture element in the beam image 360 which is on both (1) a first straight line going through the pinpointing picture elements A and B, and (2) a second straight line going through the pinpointing picture elements C and D.

The pinpointing corner picture element E (where the north east corner 362e12 of the boundary image 362e is supposed to be), the pinpointing picture elements A and B (through both of which the side 362e1 of the boundary image 362e is supposed to pass), and the pinpointing picture elements C and D (through both of which the side 362e2 of the boundary image 362e is supposed to pass) each helps determine the position of the radiation detector 100 with respect to the radiation beam 340. In general, the more pinpointing picture elements of the boundary image 362e are determined, the more accurately the position of the radiation detector 100 with respect to the radiation beam 340 is determined.

FIG. 3D is a flowchart 380 summarizing and generalizing the determination of the position of the radiation detector 100 with respect to the radiation beam 340 by determining one or more pinpointing picture elements of the boundary image 362e, according to an embodiment. Specifically, in step 382, a radiation detector (e.g., the radiation detector 100 of FIG. 3A) may be exposed to a radiation beam (e.g., the radiation beam 340 of FIG. 3A) thereby causing the radiation detector to capture a beam image (e.g., the beam image 360 of FIG. 3B) of the radiation beam. In step 384, in the beam image, M pinpointing picture elements (e.g., the pinpointing picture elements A, B, C, D, and E of FIG. 3B) of a boundary image (e.g., the boundary image 362e of FIG. 3B) of a boundary (e.g., the boundary 342 of FIG. 3A) of the radiation beam may be determined, wherein M is a positive integer (e.g., M=5 in FIG. 3B).

In an embodiment, the determinations of the pinpointing picture elements A, B, C, D, and E as described above may be performed by the radiation detector 100. In an embodiment, the boundary image 362e may be a closed line (i.e., having no end point) as shown in FIG. 3B. This happens when the entire radiation beam 340 falls on the radiation detector 100 (FIG. 3A). In an alternative embodiment, a portion of the radiation beam 340 may fall outside the radiation detector 100 as shown in FIG. 3E. As a result, with reference to FIG. 3F, the resulting boundary image 362e (which includes straight line segments PQ QR, and RS) is not a closed line and has 2 end points P and S.

In an embodiment, with reference to FIG. 3G, the imaging system 300 may further include another radiation detector 100′ similar to the radiation detector 100. In an embodiment, the radiation detector 100′ may also be exposed the radiation beam 340 thereby causing the radiation detector 100′ to capture a beam image (not shown, but similar to the beam image 360 of FIG. 3B) of the radiation beam 340. In an embodiment, one or more pinpointing picture element determinations similar to the pinpointing picture element determinations described above with respect to the radiation detector 100 may also be performed for the radiation detector 100′, thereby providing the position of the radiation detector 100′ with respect to the radiation beam 340.

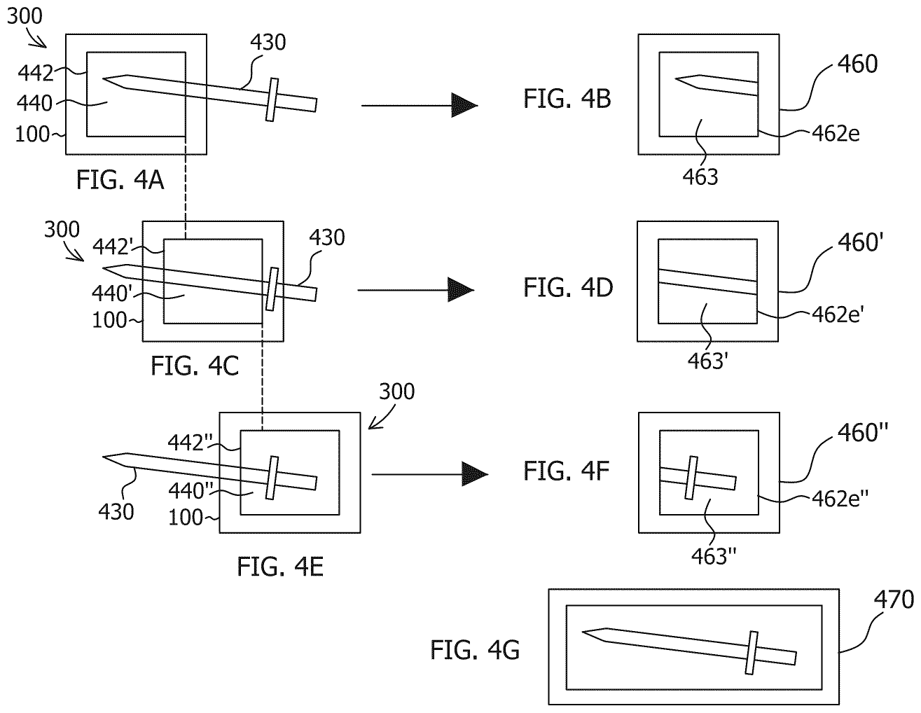

FIG. 4A-FIG. 4G schematically show an operation of the imaging system 300 of FIG. 3A, according to an alternative embodiment. An object 430 to be imaged may be a sword inside a carton box (not shown) for example; and the radiation used for imaging may be X-ray. For simplicity, only the radiation detector 100 and the radiation beams for imaging are shown in FIG. 4A, FIG. 4C, and FIG. 4E (i.e., the other parts of the imaging system 300 such as the radiation source 310 and the mask 322 are not shown). Moreover, the radiation detector 100 and the radiation beams are shown in top views in FIG. 4A, FIG. 4C, and FIG. 4E.

In an embodiment, the operation of the imaging system 300 in capturing an image of the object 430 using multiple exposures may be as follows. For the first exposure, the radiation detector 100 may be exposed to a radiation beam 440 (FIG. 4A) causing the radiation detector 100 to capture a beam image 460 which may also be called a first partial image 460 (FIG. 4B).

Next, in an embodiment, for the second exposure, the object 430 may remain stationary and the imaging system 300 (FIG. 3A) including the radiation detector 100, the radiation source 310, and the mask 320 may be moved to the right from the position as shown in FIG. 4A to the next position as shown in FIG. 4C. Then, the radiation detector 100 may be exposed to a radiation beam 440′ (FIG. 4C) causing the radiation detector 100 to capture a beam image 460′ which may also be called a second partial image 460′ (FIG. 4D).

Next, in an embodiment, for the third exposure, the object 430 may remain stationary and the imaging system 300 (FIG. 3A) including the radiation detector 100, the radiation source 310, and the mask 320 may be moved to the right from the position as shown in FIG. 4C to the next position as shown in FIG. 4E. Then, the radiation detector 100 may be exposed to a radiation beam 440″ (FIG. 4E) causing the radiation detector 100 to capture a beam image 460″ which may also be called a third partial image 460″ (FIG. 4F).

In an embodiment, with reference to FIG. 4A-FIG. 4B, during the first exposure, the position of the radiation detector 100 with respect to the radiation beam 440 may be determined by determining, in the first partial image 460, one or more pinpointing picture elements (not shown) of the boundary image 462e of the boundary 442 of the radiation beam 440. Similarly, in an embodiment, with reference to FIG. 4C-FIG. 4D, during the second exposure, the position of the radiation detector 100 with respect to the radiation beam 440′ may be determined by determining, in the second partial image 460′, one or more pinpointing picture elements (not shown) of the boundary image 462e′ of the boundary 442′ of the radiation beam 440′. Similarly, in an embodiment, with reference to FIG. 4E-FIG. 4F, during the third exposure, the position of the radiation detector 100 with respect to the radiation beam 440″ may be determined by determining, in the beam image 460″, one or more pinpointing picture elements (not shown) of the boundary image 462e″ of the boundary 442″ of the radiation beam 440″.

In an embodiment, the first partial image 460, the second partial image 460′, and the third partial image 460″ may be stitched resulting in a combined image 470 (FIG. 4G) of the object 430 based on (A) the position of the radiation detector 100 with respect to the radiation beam 440 in the first exposure, (B) the position of the radiation detector 100 with respect to the radiation beam 440′ in the second exposure, and (C) the position of the radiation detector 100 with respect to the radiation beam 440″ in the third exposure. The shapes and positions of the radiation beams 440, 440′ and 440″ are known and stitching the partial images 460, 460′ and 460″ may be further based on them. In other words, the first partial image 460, the second partial image 460′, and the third partial image 460″ may be stitched resulting in the combined image 470 (FIG. 4G) of the object 430 based on (A) the one or more pinpointing picture elements in the beam image 460 of the boundary image 462e of the boundary 442 of the radiation beam 440 in the first exposure, (B) the one or more pinpointing picture elements in the beam image 460′ of the boundary image 462e′ of the boundary 442′ of the radiation beam 440′ in the second exposure, and (C) the one or more pinpointing picture elements in the beam image 460″ of the boundary image 462e″ of the boundary 442″ of the radiation beam 440″ in the third exposure.

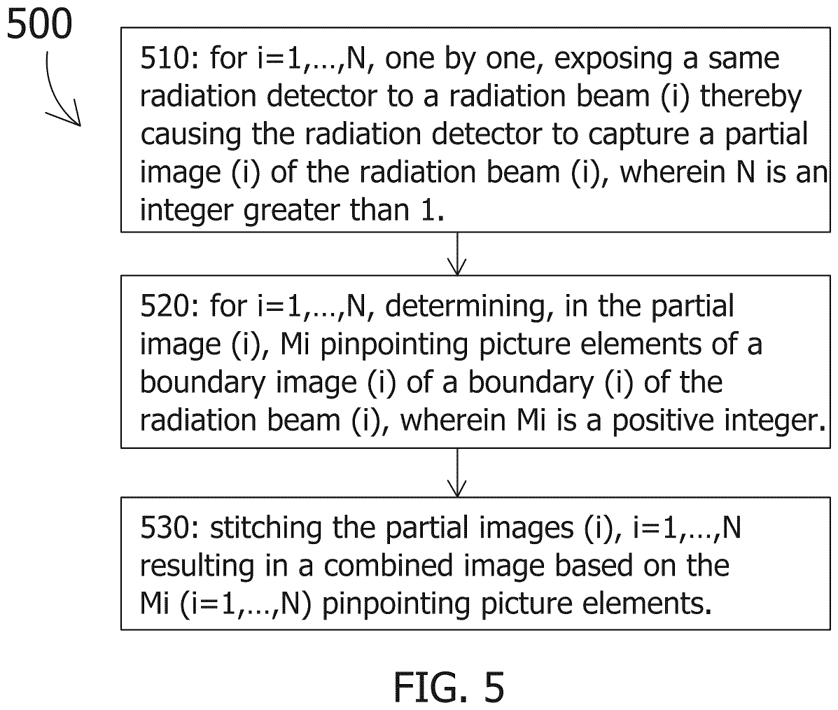

FIG. 5 shows a flowchart 500 summarizing and generalizing the operation of the imaging system 300 described above for obtaining an image of the object 430 using multiple exposures, according to an embodiment. Specifically, in step 510, for i=1, . . . , N, one by one, a same radiation detector (e.g., the radiation detector 100 of FIG. 4A) may be exposed to a radiation beam (i) (e.g., the radiation beam 440 of FIG. 4A) thereby causing the radiation detector to capture a partial image (i) (e.g., the first partial image 460 of FIG. 4B) of the radiation beam (i), wherein N is an integer greater than 1 (e.g., N=3 in FIG. 4A-FIG. 4G).

In step 520, for i=1, . . . , N, in the partial image (i) (e.g., the first partial image 460 in FIG. 4B), Mi pinpointing picture elements of a boundary image (i) (e.g., the boundary image 462e of FIG. 4B) of a boundary (i) (e.g., the boundary 442 of FIG. 4A) of the radiation beam (i) (e.g., the radiation beam 440 of FIG. 4A) may be determined, wherein Mi is a positive integer. In step 530, the partial images (i), i=1, . . . , N (e.g., the partial images 460, 460′, and 460″) may be stitched resulting in a combined image (e.g., the combined image 470 of FIG. 4G) based on the Mi (i=1, . . . , N) pinpointing picture elements.

In an embodiment, with reference to FIG. 4A-FIG. 4G, the region 463 (FIG. 4B) of the first partial image 460 bounded by the boundary image 462e may overlap the region 463′ (FIG. 4D) of the second partial image 460′ bounded by the boundary image 462e′. This may happen when the radiation beam 440′ (FIG. C) illuminates some part of the object 430 (or the scene) illuminated earlier by the radiation beam 440 (FIG. 4A).

Similarly, in an embodiment, the region 463′ (FIG. 4D) of the partial image 460′ bounded by the boundary image 462e′ may overlap the region 463″ (FIG. 4F) of the partial image 460″ bounded by the boundary image 462e″. This may happen when the radiation beam 440″ (FIG. E) illuminates some part of the object 430 (or the scene) illuminated earlier by the radiation beam 440′ (FIG. 4C).

In an embodiment, with reference to FIG. 4B, the values of some picture elements of the first partial image 460 outside the boundary image 462e as pinpointed by the one or more pinpointing picture elements of the boundary image 462e (like the picture element 365 of FIG. 3C which is outside the boundary image 362e as pinpointed by the pinpointing picture elements A, B, C, D, and E) may be used in determining the values of some picture elements of the combined image 470 (FIG. 4G). Similarly, in an embodiment, with reference to FIG. 4D, the values of some picture elements of the second partial image 460′ outside the boundary image 462e′ as pinpointed by the one or more pinpointing picture elements of the boundary image 462e′ may be used in determining the values of some picture elements of the combined image 470 (FIG. 4G). Similarly, in an embodiment, with reference to FIG. 4F, the values of some picture elements of the third partial image 460″ outside the boundary image 462e″ as pinpointed by the one or more pinpointing picture elements of the boundary image 462e″ may be used in determining the values of some picture elements of the combined image 470 (FIG. 4G).

In an alternative embodiment, with reference to FIG. 4B, the values of the picture elements of the first partial image 460 outside the boundary image 462e as pinpointed by the one or more pinpointing picture elements of the boundary image 462e are not used in determining the values of picture elements of the combined image 470 (FIG. 4G). Similarly, in an embodiment, with reference to FIG. 4D, the values of the picture elements of the second partial image 460′ outside the boundary image 462e′ as pinpointed by the one or more pinpointing picture elements of the boundary image 462e′ are not used in determining the values of picture elements of the combined image 470 (FIG. 4G). Similarly, in an embodiment, with reference to FIG. 4F, the values of the picture elements of the third partial image 460″ outside the boundary image 462e″ as pinpointed by the one or more pinpointing picture elements of the boundary image 462e″ are not used in determining the values of picture elements of the combined image 470 (FIG. 4G).

In the embodiments described above, the mask window 322 of the mask 320 (FIG. 3A) has a rectangular shape. In general, the mask window 322 may have any shape (e.g., trapezoid, etc).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method, comprising:
for i=1, . . . , N, one by one, exposing a radiation detector to a radiation beam (i) thereby causing the radiation detector to capture a partial image (i) of the radiation beam (i), wherein N is an integer greater than 1;
for i=1, . . . , N, determining, in the partial image (i), Mi pinpointing picture elements of a boundary image (i) of a boundary (i) of the radiation beam (i), wherein Mi is a positive integer; and
stitching the partial images (i), i=1, . . . , N resulting in a combined image based on the Mi (i=1, . . . , N) pinpointing picture elements.

2. The method of claim 1, wherein for i=1, . . . , N, the boundary image (i) is a closed line.

3. The method of claim 1, wherein for i=1, . . . , N, the boundary image (i) is a rectangle.

4. The method of claim 1,
wherein for i=1, . . . , N, the Mi pinpointing picture elements comprise a pinpointing picture element (i, 1), a pinpointing picture element (i, 2), a pinpointing picture element (i, 3), a pinpointing picture element (i, 4), and a pinpointing corner picture element (i), and
wherein for i=1, . . . , N, the pinpointing corner picture element (i) is on both (A) a straight line going through the pinpointing picture element (i, 1) and the pinpointing picture element (i, 2), and (B) a straight line going through the pinpointing picture element (i, 3) and the pinpointing picture element (i, 4).

5. The method of claim 1, wherein for i=1, . . . , N, the boundary image (i) is not a closed line.

6. The method of claim 1, wherein for i=1, . . . , N, intensity of radiation gradually falls when moving from inside the radiation beam (i) to outside the radiation beam (i) across the boundary (i) of the radiation beam (i).

7. The method of claim 1, wherein for i=1, . . . , N−1, a region (i) of the partial image (i) bounded by the boundary image (i) overlaps a region (i+1) of the partial image (i+1) bounded by the boundary image (i+1).

8. The method of claim 1, wherein for i=1, . . . , N, values of picture elements of the partial image (i) outside the boundary image (i) as pinpointed by the Mi pinpointing picture elements are not used in determining values of picture elements of the combined image.

9. The method of claim 1, wherein for i=1, . . . , N, values of some picture elements of the partial image (i) outside the boundary image (i) as pinpointed by the Mi pinpointing picture elements are used in determining values of picture elements of the combined image.

10. A method, comprising:
exposing a first radiation detector to a radiation beam thereby causing the first radiation detector to capture a first beam image of the radiation beam; and
determining, in the first beam image, M1 pinpointing picture elements of a first boundary image of a boundary of the radiation beam, wherein M1 is a positive integer.

11. The method of claim 10, wherein the first boundary image is a closed line.

12. The method of claim 10, wherein the first boundary image is a rectangle.

13. The method of claim 10,
wherein the M1 pinpointing picture elements comprise a first pinpointing picture element, a second pinpointing picture element, a third pinpointing picture element, a fourth pinpointing picture element, and a pinpointing corner picture element, and
wherein the pinpointing corner picture element is on both (A) a first straight line going through the first and second pinpointing picture elements, and (B) a second straight line going through the third and fourth pinpointing picture elements.

14. The method of claim 10, wherein the first boundary image is not a closed line.

15. The method of claim 10, wherein intensity of radiation gradually falls when moving from inside the radiation beam to outside the radiation beam across the boundary of the radiation beam.

16. The method of claim 10, further comprising:
exposing a second radiation detector to the radiation beam thereby causing the second radiation detector to capture a second beam image of the radiation beam; and
determining, in the second beam image, M2 pinpointing picture elements of a second boundary image of the boundary of the radiation beam, wherein M2 is a positive integer.

17. An apparatus, comprising a first radiation detector configured to (A) capture a first beam image of a radiation beam in response to the first radiation detector being exposed to the radiation beam and (B) determine, in the first beam image, M1 pinpointing picture elements of a first boundary image of a boundary of the radiation beam, wherein M1 is a positive integer.

18. The apparatus of claim 17, wherein the first boundary image is a closed line.

19. The apparatus of claim 17, wherein intensity of radiation gradually falls when moving from inside the radiation beam to outside the radiation beam across the boundary of the radiation beam.

20. The apparatus of claim 17, further comprising a second radiation detector configured to (A) capture a second image of the radiation beam in response to the second radiation detector being exposed to the radiation beam and (B) determine, in the second beam image, M2 pinpointing picture elements of a second boundary image of the boundary of the radiation beam, wherein M2 is a positive integer.
